(19) United States
(12) Patent Application Publication
(10) Pub. No.: US 2004/0019247 A1
(43) Pub. Date: Jan. 29, 2004

(54) Continuous recovery of styrene from a styrene-containing mixture

(75) Inventors: Konrad Mitulla, Ludwigshafen (DE); Heinz Friedrich Sutoris, Worms (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/456,783

(22) Filed: Sep. 5, 2003

Related U.S. Application Data
(63) Continuation of application No. 09/914,252, filed on Aug. 24, 2001, now abandoned.
Application No. 09/914,252 is a 371 of international application No. PCT/EP00/01584, filed on Feb. 25, 2000.

(30) Foreign Application Priority Data
Feb. 26, 1999 (DE) 199 084 63

(51) Int. Cl. C07C 7/00

(52) U.S. Cl. 585/807; 585/804

(57) ABSTRACT

Styrene is recovered continuously from a styrene-containing mixture by distillation of the mixture in a cascade of n distillation columns, where

(i) a feed stream comprising a stabilizer system is fed into the first column and/or a stabilizer system is introduced into at least one distillation column upstream of the nth distillation column, where the stabiliser system comprises N-oxyl radicals;

(ii) a high boiler fraction having a boiling point higher than that of styrene and comprising the stabilizer system accumulates in the bottom of the nth distillation column;

(iii) a substream of the high boiler fraction is recirculated and introduced into at least one distillation column upstream of the nth distillation column;

(iv) the remainder of the high boiler fraction is discharged from the process.

The present invention relates to a process for the continuous recovery of styrene from a styrene-containing mixture by distillation in a cascade comprising a plurality of distillation columns.

Crude styrene, i.e. a crude mixture comprising styrene and ethylbenzene, is obtained in the preparation of styrene from ethylbenzene by dehydrogenation. The pure styrene is usually recovered from this mixture by distillation. It is known that many unsaturated compounds tend to undergo free-radical polymerization when the temperature is increased. For this reason, vinylaromatic compounds such as styrene have to be stabilized by means of suitable compounds in order to prevent premature polymerization when the crude products obtained industrially are purified by distillation. These stabilizers or polymerization inhibitors are usually added to the crude products to be distilled either before or during the purification step. Despite this measure, a certain proportion of oligomers or polymers is obtained. In some cases, particularly in the event of operating malfunctions, complete polymerization of the monomer batch can occur during purification or distillation. This incurs high costs because of the thorough cleaning required and the loss of production.

The Soviet Patents SU-1027150, SU-1558888 and SU-1139722 describe the stabilization of styrene by use of nitroxyl or bisnitroxyl compounds.

WO-96/16921 discloses mixtures of vinylaromatic compounds with sterically hindered nitroxyl compounds which are activated by traces of oxygen.

JP Hei 1-165534 discloses piperidyloxy derivatives as polymerization inhibitors for styrene.

U.S. Pat. No. 5,254,760 and DE-19622498 describe mixtures of nitroxyl and nitro compounds for stabilizing vinylaromatic compounds during purification or distillation.

DE 19651307 describes mixtures comprising vinyl-containing compounds such as styrene and a mixture of an N-oxyl compound and an iron compound to inhibit premature polymerization. These mixtures are effectively stabilized against premature polymerization during purification or distillation.

To achieve sufficient stabilization against undesired polymerization, the stabilizers mentioned are used in an amount of from about 5 to 1000 ppm, based on the styrene-containing mixture. The stabilizers generally accumulate in the bottoms from the column in which the pure styrene is taken off at the top. The distillation residue, including the stabilizers dissolved therein, is generally discarded.

U.S. Pat. No. 4,272,344 describes a process for distilling vinylaromatic compounds in which 2,6-dinitro-p-cresol is used as polymerization inhibitor. It is stated that part of the distillation residue can be recirculated to the distillation system in order to minimize the required amount of stabilizer which is continually consumed during the distillation. However, when the process is carried out in practice, the opportunities for this recirculation are restricted by the fact that the distillation residue contains a high proportion of styrene polymers and therefore has a highly viscous or resin-like consistency. Recirculation is therefore restricted to very small amounts so that the concentration of styrene polymers in the distillation columns does not reach unacceptably high values. A large part of the 2,6-dinitro-p-cresol inhibitor is consumed irreversibly during the distillation by reaction with the styrene radicals which are spontaneously formed. Recirculation makes use of only the unconsumed part of the stabilizer. Reactivation to form new or previously present species which are effective as free-radical traps from the consumed stabilizer does not take place. Since the residual content of active stabilizer in the recirculated distillation residue can fluctuate in the process of U.S. Pat. No. 4,272,344, the addition of sufficient amounts of fresh stabilizer is necessary to ensure effective stabilization. Overall, little stabilizer is saved by recirculation in the process of U.S. Pat. No. 4,272,344.

It is an object of the present invention to provide a process for the continuous recovery of styrene from a styrene-containing mixture by distillation in the presence of a stabilizer, in which process the stabilizer is utilized as effectively as possible.

We have found that this object is achieved by means of N-oxyl radicals which are effective polymerization inhibitors and have surprisingly been found to be capable of activation or reactivation and to be able to be recirculated to the distillation system to a greater extent than is possible in the case of other stabilizers.

The present invention accordingly provides a process for the continuous recovery of styrene from a styrene-containing mixture by distillation of the mixture in a cascade of n distillation columns, where

(i) a feed stream comprising a stabilizer system is fed into the first column and/or a stabilizer system is introduced into at least one distillation column upstream of the nth distillation column, where the stabilizer system comprises N-oxyl radicals;

(ii) a high boiler fraction having a boiling point higher than that of styrene and comprising the stabilizer system accumulates in the bottom of the nth distillation column;

(iii) a substream of the high boiler fraction is recirculated and introduced into at least one distillation column upstream of the nth distillation column;

(iv) the remainder of the high boiler fraction is discharged from the process.

The styrene-containing mixture used in the process of the present invention is generally a product mixture, as a rule one obtained industrially, from which styrene can be isolated by distillation. A preferred example is crude styrene, i.e. a crude mixture obtained in the production of styrene from ethylbenzene and comprising, in addition to styrene and ethylbenzene, subordinate amounts of toluene, benzene, cumene and/or α-methylstyrene. In addition, crude styrene further comprises, typically in an amount up to 3% by weight, e.g. from 0.5 to 1.2% by weight, based on styrene, constituents having a boiling point higher than that of styrene (known as higher boilers), for example stilbenes, styrene oligomers and styrene polymers and also diphenylethane and 2-phenylnaphthalene.

Typical mixtures from which styrene can be recovered by the process of the present invention have, for example, the following composition: 1% of benzene, 2% of toluene, 40% of ethylbenzene, 56% of styrene and 1% of higher boilers.

Owing to the close proximity of the boiling points of styrene and ethylbenzene (145° C. and 136° C., respectively, at atmospheric pressure) and the high purity demanded of styrene, its isolation in pure form requires a high separation efficiency in the distillation. According to the invention, purification is carried out by distillation in a cascade of n distillation columns, where the bottom product from a distillation column is in each case fed into the next downstream distillation column. The feed point is preferably in the region of the middle of the column. The styrene-containing mixture is fed as feed stream into the first column. The parameter n is a positive integer≧2 and indicates the number of distillation columns in the cascade. In general, it is preferred that n is from 2 to 4, e.g. 2 or 3. In the nth distillation column, pure styrene is generally taken off at the top, while the constituents of the crude styrene having boiling points lower than that of styrene are taken off at the top in the distillation columns upstream of the nth column. The bottom product from the nth column can be passed to a concentrator, e.g. a thin film evaporator or a flash evaporator, to isolate residual amounts of styrene and/or methylstyrenes. The low boiler fraction obtained in this way can be further fractionated in a work-up column. The arrangement and connection of the individual distillation columns for carrying out the process of the present invention can be readily determined by a person skilled in the art on the basis of his expert judgement.

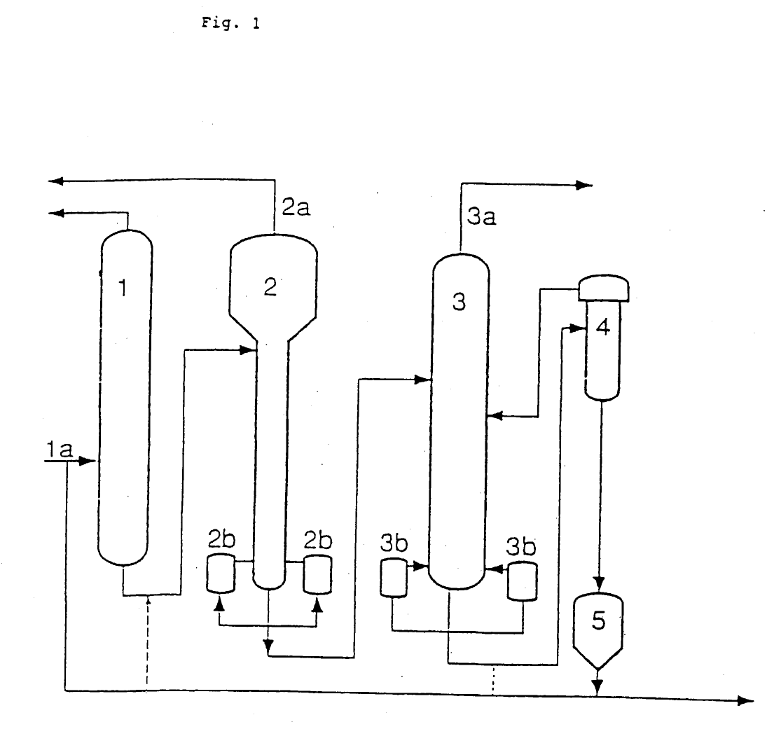

A typical arrangement for the industrial distillation of styrene is described in the Kunststoff-Handbuch, Volume 4 (Polystyrol), Section 2.3.1.4, 30 ff. (Munich 1996). A distillation plant which can be used according to the present invention is shown in FIG. 1 and can comprise, for example, a benzene (toluene) column 1 to which a mixture of, for example, essentially styrene, ethylbenzene, benzene and toluene 1a is fed, an ethylbenzene column 2, which serves for the separation and recovery of ethylbenzene 2a and the styrene column 3 from which the pure styrene 3a is finally recovered. The ethylbenzene column 2 and styrene column 3 are each provided with boilers 2b or 3b, i.e. they have a heatable bottom.

According to the present invention, for example, a substream is taken from the bottom of the column 3 and added to the feed stream to column 1 and/or 2. In a preferred embodiment, the bottom product from column 3 is passed to the facility consisting essentially of the equipment items 4 and 5. Here, 4 is a concentrator configured, for example, as a thin film evaporator or flash evaporator in which the product stream taken from the bottom of column 3 is freed of low boilers. The low boilers can be further separated into styrene and α-(β-)methylstyrene in a work-up column (not shown). A substream of the concentrate obtained from 4 and subjected to intermediate storage in 5 is then recirculated.

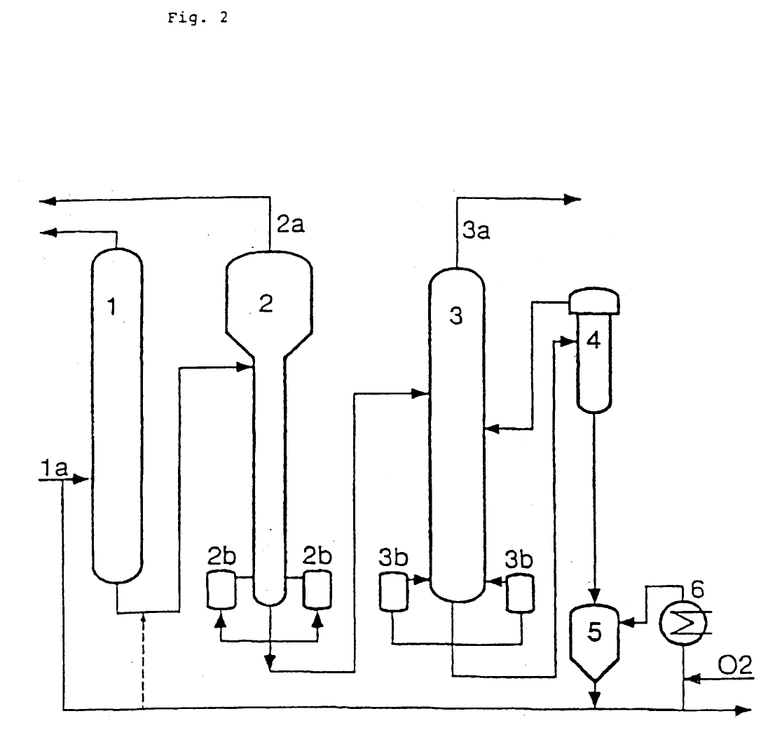

FIG. 2 shows an extended distillation plant in which the concentrate of the high boiler fraction is treated with oxygen, as per a preferred embodiment of the process of the invention. A temperature suitable for the activation can be set in the heat exchanger 6.

According to the present invention, the distillation of the styrene-containing mixture is carried out in the presence of a stabilizer system comprising N-oxyl radicals. The N-oxyl radicals are stable free radicals which have hitherto also been referred to as persistent radicals. They possess one or more unpaired electrons. They can generally be prepared as a pure substance and can be stored without decomposition for a number of years. They themselves are not able to trigger a free-radically initiated polymerization. They eagerly react with and trap organic free radicals which are formed spontaneously, for example, in the distillation of ethylenically unsaturated compounds. The N-oxyl radicals are generally sterically hindered, i.e. they are derived from a secondary amine whose hydrogen atoms in the a position relative to the nitrogen atom bearing the oxyl group have all been replaced, for example, by alkyl groups.

In addition to the N-oxyl radicals, the stabilizer system may further comprise other components such as the polmerisation retarders or activators described below.

Suitable N-oxyls have, for example, the following structures

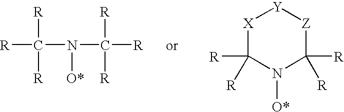

where R are identical or different alkyl, cycloalkyl, aralkyl or aryl radicals having up to 24 carbon atoms, where geminal R radicals can also be connected pairwise to form a ring system, and X, Y and Z are, independently of one another, CR′₂, CR′OH, CR′(COOH), O, S, CO or a chemical bond, with the proviso that at most one radical X, Y or Z is O or S and at most one radical X, Y or Z is a chemical bond. R′ is hydrogen or an alkyl, cycloalkyl, aralkyl or aryl radical having up to 24 carbon atoms. For example, R is a C₁-C₂₀-, in particular C₁-C₈-alkyl radical, a C₅- or or C₆-cycloalkyl radical, a benzyl radical or a phenyl radical. X-Y-Z is, for example, —(CH₂)₂— or —(CH₂)₃—, —CH₂—CH(OH)—CH₂—, —CH₂—CO—O— or —CH₂—O—.

Further suitable N-oxyl radicals are those having aromatic substituents, for example the following structures

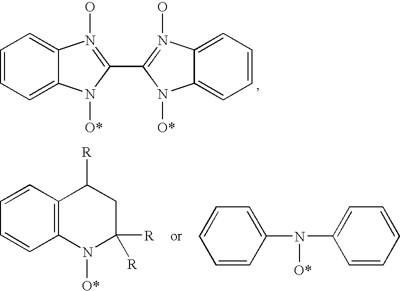

where each of the aromatic rings may additionally bear from 1 to 3 inert substituents such as C₁-C₄-alkyl, C₁-C₄-alkoxy, ester, amide or cyano.

Preference is given to using N-oxyl radicals which are derived from cyclic amines, e.g. from piperidine or pyrrolidine compounds, which may contain a further heteroatom such as nitrogen, oxygen or sulfur in the ring, where this heteroatom is not adjacent to the amine nitrogen. The steric hindrance is produced by substituents in the two positions adjacent to the amine nitrogen, with suitable substituents being hydrocarbon radicals which replace all 4 hydrogen atoms of the α-CH₂ groups. Examples of substituents are phenyl, C₃-C₆-cycloalkyl, benzyl and, in particular, C₁-C₆-alkyl radicals, where the alkyl radicals bound to the same a-carbon atom may also be joined to one another to form a 5- or 6-membered ring. N-oxyls of sterically hindered amines which are preferably used are derivatives of 2,2,6,6-tetraalkylpiperidine.

Preferred N-oxyl compounds are those of the formula (II) or (II′)

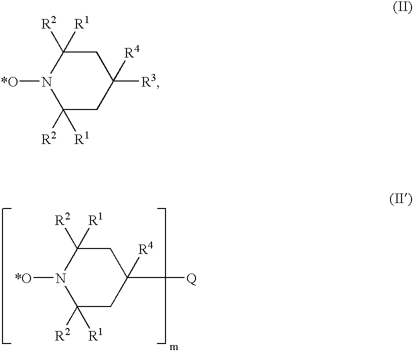

where

R¹ and R² are each, independently of one another, C₁-C₄-alkyl or phenyl or R¹ and R² together with the carbon atom to which they are bound form a 5- or 6-membered, substituted or unsubstituted, saturated hydrocarbon ring which may contain 1 or 2 heteroatoms selected from among O, S or N and also 1 or 2 keto groups,

R³ is hydrogen, hydroxy, amino, SO₃H, SO₃M, PO₃H₂, PO₃HM, PO₃M₂, organosilicon radicals or a monovalent organic radical bound via carbon, oxygen or nitrogen and preferably having from 1 to 36 atoms, where M is an alkali metal, preferably Li, Na or K,

R⁴ is hydrogen, C₁-C₁₂-alkyl, C₁-C₁₂-alkoxy

or R³ and R⁴ together are oxygen

or R³ and R⁴ together with the carbon atom to which they are bound form a 5- or 6-membered, substituted or unsubstituted, saturated ring which may contain 1 or 2 heteroatoms selected from among O, S or N and also 1 or 2 keto groups,

Q is an m-valent organic radical bound via carbon, oxygen or nitrogen and preferably having from 2 to 10,000 atoms, in particular from 4 to 2000 atoms,

m 2 to 100, preferably 2 or 3.

R¹ and R² can be C₁-C₄-alkyl groups such as methyl, ethyl, n-propyl, isopropyl, n-butyl, isobutyl, sec-butyl or tert-butyl or they can together form a tetramethylene or pentamethylene group. R¹ and R² are preferably methyl groups.

Examples of suitable radicals R⁴ are hydrogen, the abovementioned C₁-C₄-alkyl groups and also pentyl, sec-pentyl, tert-pentyl, neopentyl, 2,3-dimethylbut-2-yl, hexyl, 2-methylpentyl, heptyl, 2-methylhexyl, 2-ethylhexyl, octyl, isooctyl, 2-ethylhexyl, nonyl, 2-methylnonyl, isononyl, 2-methyloctyl, decyl, isodecyl, 2-methylnonyl, undecyl, isoundecyl, dodecyl and isododecyl.

Preferred radicals R³ are hydrogen,

C₁-C₂₀-alkyl groups such as methyl, ethyl, n-propyl, isopropyl, n-butyl, isobutyl, pentyl,

hydroxy,

C₂-C₂₀-alkoxy groups such as methoxy, ethoxy, propoxy, and t-butoxy,

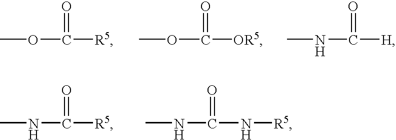

where R⁵ is C₁-C₁₂-alkyl, C₆-C₁₂-aryl or C₇-C₁₄-aralkyl, and also organosilicon radicals of the formula

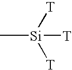

where the groups T can be identical or different and are C₁-C₁₂-alkyl or phenyl.

Examples of such organosilicon radicals are —Si(CH₃)₃ and —Si(C₂H₅)₃.

R³ and R⁴ together with the carbon atom to which they are bound can represent, for example,

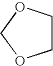

Preferred radicals Q are, for example, the following groups

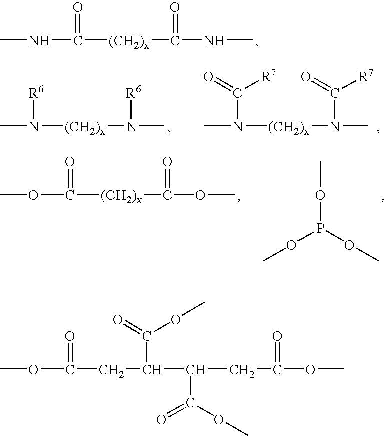

where

R⁶ is C₁-C₁₂-alkyl,

R⁷ is hydrogen or C₁-C₁₈-alkyl,

x is from 1 to 12

Further suitable N-oxyls also include oligomeric or polymeric compounds which have a polysiloxane as main polymer chain and are substituted in the side chain by N-oxyl groups derived from 2,2,6,6-tetraalkylpiperidine. Here, the preferred N-oxyl group is the 2,2,6,6-tetramethylpiperidin-N-oxyl group. Examples of such N-oxyls which can likewise be used according to the present invention may be found in WO 69/17002. Furthermore, this publication gives examples of syntheses of the amino compounds on which the N-oxyls are based.

Further N-oxyl radicals which are suitable according to the present invention are the N-oxyl radicals mentioned in DE 19651307 as a constituent of the mixture disclosed there. The full contents of this publication are hereby incorporated by reference.

Preferred nitroxyl compounds are the following:

1-oxyl-2,2,6,6-tetramethylpiperidine,

1-oxyl-2,2,6,6-tetramethylpiperidin-4-ol,

1-oxyl-2,2,6,6-tetramethylpiperidin-4-one,

1-oxyl-2,2,6,6-tetramethylpiperidin-4-yl acetate,

1-oxyl-2,2,6,6-tetramethylpiperidin-4-yl 2-ethylhexanoate,

1-oxyl-2,2,6,6-tetramethylpiperidin-4-yl stearate,

1-oxyl-2,2,6,6-tetramethylpiperidin-4-yl benzoate,

1-oxyl-2,2,6,6-tetramethylpiperidin-4-yl (4-tert-butyl)benzoate,

bis(1-oxyl-2,2,6,6-tetramethylpiperidin-4-yl) succinate,

bis(1-oxyl-2,2,6,6-tetramethylpiperidin-4-yl) adipate,

bis(1-oxyl-2,2,6,6-tetramethylpiperidin-4-yl) sebacate,

bis(1-oxyl-2,2,6,6-tetramethylpiperidin-4-yl) n-butylmalonate,

bis(1-oxyl-2,2,6,6-tetramethylpiperidin-4-yl) phthalate,

bis(1-oxyl-2,2,6,6-tetramethylpiperidin-4-yl) isophthalate,

bis(1-oxyl-2,2,6,6-tetramethylpiperidin-4-yl) terephthalate,

bis(1-oxyl-2,2,6,6-tetramethylpiperidin-4-yl) hexyhydroterephthalate,

N,N′-bis(1-oxyl-2,2,6,6-tetramethylpiperidin-4-yl)adipinamide,

N-(1-oxyl-2,2,6,6-tetramethylpiperidin-4-yl) caprolactam,

N-(1-oxyl-2,2,6,6-tetramethylpiperidin-4-yl) dodecylsuccinimide,

2,4,6-tris[N-butyl-N-(l-oxyl-2,2,6,6,-tetramethylpiperidin-4-yl]s-triazine,

N,N′-bis(1-oxyl-2,2,6,6-tetramethylpiperidin-4-yl)-N,N′-bis-formyl-1,6-diaminohexane,

4,4′-ethylenebis(1-oxyl-2,2,6,6-tetramethylpiperazin-3-one) and tris(2,2,6,6-tetramethyl-1-oxyl-piperidin-4-yl) phosphite.

The N-oxyl radicals used according to the present invention can be prepared via various synthesis steps known per se. A preferred method of preparation employs the oxidation of a secondary amine whose NH group is converted by oxidation into the corresponding N-oxyl group. Suitable oxidizing agents are peroxides such as H₂O₂, t-butyl hydroperoxide, cumene hydroperoxide, peracids such as metachloroperbenzoic acid, α-chloroperbenzoic acid, peracetic acid, paranitroperbenzoic acid, perbenzoic acid or magnesium monoperoxyphthalate. The oxidation can be carried out in an inert solvent such as CH₂Cl₂, petroleum ether, toluene, xylene or benzene.

The parent secondary amines are either known from the literature or can readily be prepared by a person skilled in the art of organic chemical synthesis by modification of methods known per se. DE 19651307 discloses the preparation of various N-oxyl radicals which are suitable for use according to the present invention.

As feed stream, the styrene-containing mixture is fed into the first distillation column. This styrene-containing mixture can already have been admixed with a stabilizer system comprising N-oxyl radicals. This is normally the case when, for example in the production of the styrene-containing mixture, a styrene-containing gaseous reaction mixture is condensed using a stabilizer-containing crude liquid mixture. Alternatively or additionally, the stabilizer system can be introduced into at least one distillation column upstream of the nth distillation column. The stabilizer system can advantageously be mixed into the feed to a distillation column or else be introduced into the bottom of the column.

The N-oxyl radicals and the optional components of the stabilizer system are relatively nonvolatile compounds. For this reason, a high boiler fraction comprising the stabilizer system accumulates in the bottoms of the nth distillation column. In general, the high boiler fraction comprises the relatively high-boiling constituents of the styrene-containing mixture and/or styrene oligomers which are formed to a small extent during the distillation. In particular cases, a solvent which has a boiling point higher than that of styrene and accumulates in the high boiler fraction so as to serve as carrier for the stabilizer system can also be mixed into the styrene-containing mixture prior to the distillation.

According to the present invention, a substream of the solution of the stabilizer system in the high boiler fraction which accumulates at the bottom of the nth distillation column is recirculated and added to the feed to at least one distillation column upstream of the nth distillation column. The recirculated stream can be divided and added at a plurality of points, e.g. to the feed to the first column and to the feed to the second column. In the preferred case of a distillation in three successive distillation columns, preference is given to adding from 50 to 100% by weight of the recirculated high boiler fraction to the feed to the first distillation column and from 0 to 50% by weight of the recirculated high boiler fraction to the feed to the second distillation column. The recirculated stabilizer solution is advantageously mixed into the feed to an upstream distillation column; however, the recirculated solution can also be introduced directly into the bottom of an upstream distillation column.

In general, the high boiler fraction taken from the bottom of the nth distillation column is preferably concentrated, i.e. freed of low boilers, prior to recirculation or discharge. Examples of apparatuses suitable for this purpose are thin film evaporators or flash evaporators. The low boiler fraction obtained here can be further fractionated into styrene and α- or β-methylstyrene in a work-up column. The α-methylstyrene content of the recirculated stabilizer solution is preferably less than 3% by weight, e.g. from 0.01 to 2% by weight. Higher proportions of α-methylstyrene in the recirculated stabilizer solution can in some circumstances lead to the α-methylstyrene content of the pure styrene fraction taken off at the top of the nth distillation column rising to an undesirable degree. Concentrating the high boiler fraction as indicated above prior to recirculation enables the α-methylstyrene content to be readily lowered to the specified values. After concentration, the concentration of N-oxyl radicals in the high boiler fraction is generally from 0.2 to 100 g/l.

A fresh amount of stabilizer system comprising N-oxyl radicals is introduced discontinuously or continuously together with the feed to the first column or by addition to one of the columns in order to replace the amount of stabilizer system removed from the system in the substream of high boiler fraction discharged from the bottom of the nth distillation column. The supplementary amount of N-oxyl radicals and possibly further components of the stabilizer system can be added as such or in the form of a solution in a solvent such as water, C₁-C₆-alkanols such as methanol, ethanol, propanol or n-, i- or t-butanol, if desired in a mixture with water, ketones such as acetone, methyl ethyl ketone, methyl propyl ketone or methyl butyl ketone, diols such as glycol or propylene glycol, or their monoalkyl or dialkyl ethers, oligomeric or polymeric ethylene glycols and propylene glycols or their alkyl ethers, diamines such as ethylene diamine or propylene diamine or their monoalkylimino or dialkylimino derivatives, oligomeric or polymeric ethylene diamines or their alkylimino derivatives. However, the styrene-containing mixture to be purified is preferably used as solvent or suspension medium for the stabilizer system. Thus, the mixture obtained in the dehydrogenation of ethylbenzene, which consists predominantly of styrene, ethylbenzene, toluene and further substituted aromatics, can be used for this purpose. The solution of the stabilizer system is advantageously introduced into the feed to a distillation column upstream of the nth distillation column, e.g. mixed with this. Thus, for example, continuous metering of fresh N-oxyl radical solution into the feed to the first and/or second distillation column can be provided.

The N-oxyl radicals are preferably used in such an amount that the concentration of N-oxyl radicals in the bottom of each distillation column is at least 0.1 ppm, in particular from 1 to 500 ppm, preferably from 5 to 150 ppm. The amount in the bottom of a distillation column is made up of the recirculated and freshly added amounts of N-oxyl radical.

The N-oxyl radicals used according to the present invention are effective inhibitors of styrene polymerization and strongly suppress the formation of styrene polymers during the distillation. The high boiler fraction in the nth column therefore has, even after concentration, a desirably low viscosity, so that relatively large substreams can be recirculated without problems. The temperature in the bottom of the nth distillation column is generally higher than that in the bottom sections of the upstream columns, since fractions having boiling points lower than that of styrene are distilled off in the upstream columns while styrene is taken off at the top of the nth column. It is assumed that partial reactivation of the N-oxyl radicals takes place in the bottom of the nth distillation column. The reactivation can be depicted by the following scheme:

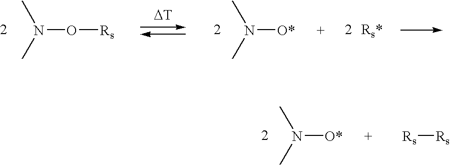

where R_{S} is an organic group comprising one or more styrene radicals. The bond between the group R_{S} and the oxygen atom of the nitroxyl radical can be reversibly broken at elevated temperature. At elevated temperature, there is, in an equilibrium reaction, a steady-state concentration of free R_{S} radicals which can combine in pairs so as to liberate the nitroxyl radicals again.

As a measure of the recirculation according to the present invention of N-oxyl radicals which are present in the recirculated stream of high boiler fraction, it is possible to define the number of cycles Z for which the N-oxyl radicals pass, on average, through the (n−1)th distillation column. The number of cycles Z is linked via the following equation to the proportion x of recirculated high boiler fraction, based on the total amount of high boiler fraction, obtained in the bottom of the nth distillation column:

$$z = \frac{1}{1 - x}$$

Preferably, the N-oxyl radicals pass through the (n−1)th distillation column an average of at least 1.4 times, preferably 2.0 times, in particular 2.5 times, particularly preferably 3 times. The numbers of cycles stated generally correspond to proportions of more than 0.3, preferably more than 0.5, in particular more than 0.6, particularly preferably more than 0.67, of the recirculated stabilizer solution. In general, preference is given to recirculating from 10 to 90% by weight, preferably from 30 to 85% by weight, in particular from 50 to 80% by weight, of the high boiler fraction obtained in the bottom of the nth distillation column.

ESR studies have shown that particularly good reactivation of the recirculated stabilizer solution can be achieved if the substream is heated to above 130° C. prior to recirculation. In a preferred embodiment of the process of the present invention, the substream of the solution of the stabilizer system is therefore heated to above 130° C., in particular 135-160° C., prior to recirculation. Heating can advantageously be carried out for a period of from 1 to 300 minutes, preferably from 10 to 60 minutes.

In a further preferred embodiment of the process of the present invention, the stabilizer system further comprises at least one polymerization retarder. Polymerization retarders are substances which do not completely suppress free-radically initiated polymerization of the styrene monomers but reduce the polymerization rate. Combining the N-oxyl radicals to be used according to the present invention with at least one polymerization retarder has the advantage that if the concentration of N-oxyl radicals drops below a threshold value required for effective inhibition, for instance in the case of a production malfunction, no sudden polymerization of the monomers present in the system occurs. Rather, a slow rise in the oligomer or polymer content occurs, so that countermeasures can be taken if necessary. The combination of the N-oxyl radicals with a polymerization retarder also displays a synergistic effect, i.e. the different mechanisms of action supplement one another so that a higher polymerization-inhibiting effect is achieved at the same total concentration of stabilizer system when using a combination of N-oxyl radicals with a polymerization retarder than is achieved when using N-oxyl radicals alone or polymerization retarders alone. The polymerization retarder is preferably used in an amount of from 50 to 2000 ppm, based on styrene. The weight ratio of N-oxyl radicals to polymerization retarder is preferably in a range from 1:20 to 20:1.

Suitable polymerization retarders are, in particular, aromatic nitro compounds, in particular those of the formula III

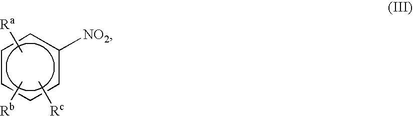

where

R^{a}, R^{b} and R^{c} are each, independently of one another, hydrogen, C₁-C₆-alkyl, halogen or a radical of the formula CN, SCN, NCO, OH, NO₂, COOH, CHO, SO₂H or SO₃H,

where the aromatic ring may be benzo-fused.

Examples of suitable compounds are

1,3-dinitrobenzene, 1,4-dinitrobenzene,

2,6-dinitro-4-methylphenol, 2-nitro-4-methylphenol,

2,4,6-trinitrophenol, 2,4-dinitro-1-naphthol,

2,4-dinitro-6-methylphenol, 2,4-dinitrochlorobenzene,

2,4-dinitrophenol, 2,4-dinitro-6-sec-butylphenol,

4-cyano-2-nitrophenol or 3-iodo-4-cyano-5-nitrophenol. Preference is given to using aromatic nitro compounds such as 2,6-dinitro-4-methylphenol, 2-nitro-4-methylphenol,

2,4-dinitro-6-sec-butylphenol or 2,4-dinitro-6-methylphenol.

The stabilizer system in the process of the present invention may further comprise one or more costabilizers selected from the group consisting of aromatic nitroso compounds, phenothiazines, quinones, hydroquinones and their ethers, phenols and their ethers, hydroxylamines and phenylenediamines.

Further suitable costabilizers are substituted phenols or hydroquinones, for example the following:

4-tert-butylcatechol, methoxyhydroquinone,

2,6-di-tert-butyl-4-methylphenol, n-octadecyl

β-(3,5-di-tert-butyl-4-hydroxyphenyl)propionate,

1,1,3-tris(2-methyl-4-hydroxy-5-tert-butylphenyl)butane,

1,3,5-trimethyl-2,4,6-tris(3,5-di-tert-butyl-4-hydroxybenzyl)-benzene, 1,3,5-tris(3,5-di-tert-butyl-4-hydroxybenzyl) isocyanurate, 1,3,5-tris[β-(3,5-di-tert-butyl-4-hydroxyphenyl) propionyl-oxyethyl isocyanurate, 1,3,5-tris(2,6-dimethyl-3-hydroxy-4-tert-butylbenzyl)isocyanurate or pentaerythrityl tetrakis[β-(3,5-di-tert-butyl-4-hydroxyphenyl)propionate].

In a preferred embodiment of the process of the present invention, the stabilizer system further comprises an activator in addition to the N-oxyl radicals used according to the present invention. An activator is a chemical compound which can increase the effect of the N-oxyl radicals by, for example, catalyzing combination reactions of free radicals.

The activator is preferably used in an amount of from 0.01 to 20% by weight, based on the N-oxyl radicals.

Suitable activators are, in particular, iron compounds or other transition metal compounds, particularly those which can be present in various oxidation states.

Preferred iron compounds suitable as activators are selected from the group consisting of

a) iron carbonyls and carbonylferrates,

b) organometallic iron carbonyl compounds,

c) unsubstituted and substituted ferrocene compounds,

d) iron compounds having ligands which contain, as donor atoms, oxygen, nitrogen, sulfur or phosphorus or a mixture of these,

e) iron halide and iron pseudohalide compounds.

Examples of compounds of group a) are iron pentacarbonyl Fe(CO)₅, diiron nonacarbonyl Fe₂(CO)₉, triiron dodecacarbonyl Fe₃(CO)₁₂ and hexairon octadecacarbonyl Fe₆(CO)₁₈, which are all soluble in slightly polar or nonpolar media. Further examples which may be mentioned are the carbonylferrates such as M₂Fe(CO)₄, M₂Fe₂(CO)₈ and M₂Fe₃(CO)₁₁, where M is one equivalent of an alkali metal or alkaline earth metal. Preference is given to using the corresponding Na compounds.

Organometallic iron carbonyl compounds of group b) are, for example, compounds of the formula

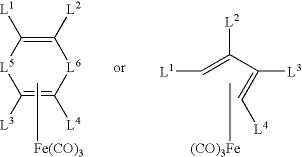

where the variables have the following meanings:

L¹-L⁴ are hydrogen, C₁-C₄-alkyl such as methyl, ethyl, propyl or t-butyl

L⁵, L⁶ are —(CH₂)ₙ— or —CO—, where n in L⁵ and L⁶ is independently 0, 1, 2 or 3.

Examples of suitable compounds are:

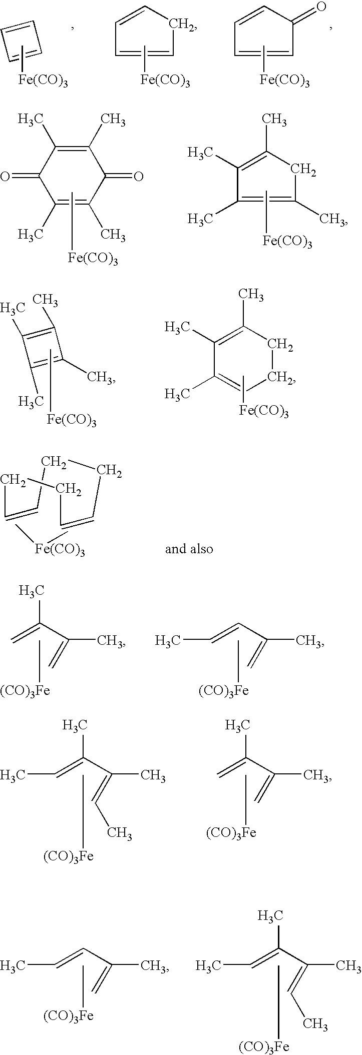

Further compounds of this group which can be used according to the present invention are binuclear Fe compounds such as [H₅C₅Fe(CO)₂]₂, [(H₃C)₅C₅Fe(CO)₂]₂ and the ferrates M[Fe(CO)₂C₅H₅] and M[Fe(CO)₂(H₃C)₅C₅] derived therefrom, where, as above, M is one equivalent of an alkali metal or alkaline earth metal and preference is given to using the corresponding Na compounds.

The compounds of group c) to be used according to the present invention include ferrocene itself and ferrocene derivatives substituted on one or both cyclopentadienyl rings. It is also possible to use dimeric ferrocene derivatives. Here, the individual ferrocene units are linked via one carbon atom of each cyclopentadienyl ring by means of a chemical bond or a methylene, ethylene, propylene, butylene or phenylphosphine bridge.

Possible substituents of the cyclopentadienyl rings are C₁-C₄-alkenyl radicals, C₇-C₁₀-aroyl, C₁-C₄-alkyl radicals such as methyl, ethyl, n-propyl, i-propyl, n-butyl, i-butyl, sec-butyl or tert-butyl. Furthermore, one or two CH₂ or CH₃ groups in these substituents can be replaced by O, NH, NCH₃ or OH, NH₂. These heteroatoms or heteroatom-containing moieties are bound to carbon atoms. It is also possible for one or two CH₂ groups to be replaced by CO or for one or two CH₃ groups to be replaced by CN. Furthermore, it is possible for diphenylphosphino radicals to function as substituents on the cyclopentadienyl rings, if desired in addition to the abovementioned groups.

Examples of ferrocene derivatives which can be used according to the present invention are

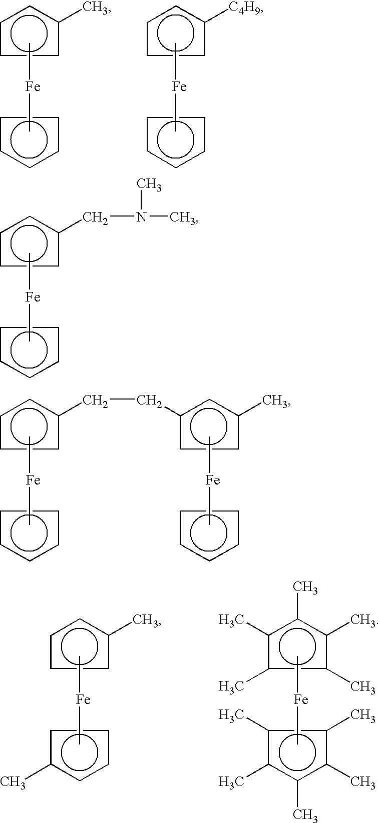

As compounds of group d), it is possible to use, for example, complexes or salts of Fe(II)/Fe(III) with O-containing ligands such as sulfate, acetate, oxalate, citrate, tartrate, lactate, gluconate or acetylacetonate (acac), i.e. compounds such as

[Fe₃O(SO₄)₆(OH)₃]^{5⊖}, [Fe₃O(O₂CCH₃)₆(OH₂)₃]^{⊕},

[Fe₃O(O₄C₂)₆(OH₂)₃]^{5⊖}, [Fe(C₄H₄O₆)₂]^{2⊖/⊖}, Fe(C₄H₄O₆),

Fe₂(C₄H₄O₆)₃, Fe(C₃H₅O₃)₂, Fe(C₆H₁₁O₇)₂, [Fe(C₂O₄)^{3⊖}],

FeC₂O₄, [Fe(C₂O₄)₂]^{2⊖}, Fe(acac)₃, Fe(acac)₂, Fe(C₆H₆O₇),

Fe(C₆H₅O₇).

Further exclusively or predominantly O-containing ligands for Fe(II) or Fe(III) may also be cyclic polyethers such as spherands, cryptands, cryptaspherands, hemispherands, coronands or open-chain representatives of these ethers as well as podands.

It is also possible to use complexes having N-containing chelating ligands such as ethylenediamine (en), 1,10-phenanthroline (phen), 1,8-naphthopyridine (napy), 2,2′-bipyridyl (bipy) and dibenzo[b,i]-1,4,8,11-tetraaza-(14)annulene (taa), i.e. compounds such as

[Fe(en)(H₂O)₄]^{2⊕/3⊕}, [Fe(en)₂(H₂O)₂]^{2⊕/3⊕},

[Fe(en)₃]^{2⊕/3⊕}, [Fe(phen)₃]^{2⊕/3⊕}, [Fe(napy)₄]^{2⊕/3⊕},

[Fe(bipy)4]^{2⊕/3⊕} and

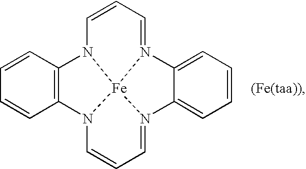

as well as complexes of iron with various, substituted porphyrin ligands, as are known from the literature (for example B. Mennier, Chem. Rev., Vol 92 (8), pp. 1411-1456, 1992). Other N-containing ligands are phthalocyanine and derivatives thereof, for example

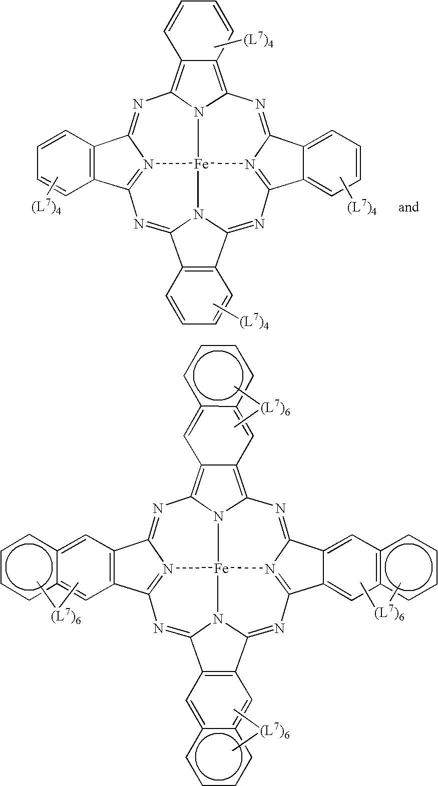

The radicals L⁷ can be, independently of one another, hydrogen, halogen, SO₃H, SO₂NH₂, SO₂NH(C₁-C₁₂-alkyl), SO₂N(C₁-C₁₂-alkyl)₂, CONH₂, CONH(C₁-C₁₂-alkyl), CON(C₁-C₁₂-alkyl)₂, cyano, hydroxy, C₁-C₁₂-alkyl, C₁-C₁₂-alkoxy or C₁-C₁₂-alkylthio. Preferred halogens are Cl and Br.

N,O-containing ligands such as ethylenediaminetetraacetic acid (EDTA) or nitrilotriacetic acid (NTA) give compounds such as

[Fe(EDTA)(H₂O)]^{⊖/2⊖}, [Fe(NTA)(H₂O)₂] or [Fe(NTA)(H₂O)₂]^{⊖},

and 8-hydroxyquinoline (quin) or 5-methyl-8-hydroxyquinoline (H₃C-quin) give compounds such as

[Fe(quin)₃]/[Fe(quin)₃]^{2⊖} or [Fe(H₃C-quin)₃]/[Fe(H₃C-quin)₃]^{2⊖},

which can likewise be used.

Further Fe compounds which may be used according to the present invention are Fe complexes with Schiff bases of salicyl aldehydes.

The preparation of these N,O-containing ligands is known and is generally carried out by condensation of aromatic or heteroaromatic α-hydroxyaldehydes with an aliphatic or aromatic diamine or polyamine. The ligands are subsequently reacted with an Fe salt in aqueous solution.

Further Fe compounds which can be used are those having S-containing ligands, for example

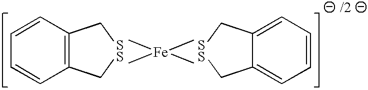

or [Fe₄S₄(SR)₄]^{4⊖/3⊖}, and also complexes of Fe(II)/Fe(III) with dithiocarbamates R₂NCS₂^{⊖} such as [Fe(S₂CNR₂) 3]^{⊖} (R═CH₃, C₂H₅).

It is also possible to use compounds of group e). Among the Fe halides, preference is given to using the Fe(II) and Fe(III) salts of Cl and Br, and also the complexes FeX₄^{⊖/2⊖} (X═Cl,Br). The Fe pseudohalide compounds to be used according to the present invention include, for example, [Fe(CN)₆]^{3⊖}/[Fe(CN)₆]^{4⊖} and also thiocyanate complexes of the series [Fe(SCN)_{3-X}(H₂O)_{3+X}]^{X⊕} (x=0, 1, 2).

As counterions for all negatively charged complex ions mentioned, preference is given to using H^{⊕}, Na^{⊕}, K^{⊕} and ammonium ions NH₄^{⊕} and also N(CH₃)₄^{⊕}, and in the case of hexacyanoferrates not only K^{⊕} but also Fe^{2⊕} in the case of [Fe(CN)₆]^{3⊖} and Fe^{3⊕} in the case of [Fe(CN₆)]^{4⊖}.

In the case of the positively charged complex ions mentioned, counterions used are preferably Cl^{⊖}, Br^{⊖}, r^{⊖}, SO₄^{2⊖}, H₃CCO₂^{⊖}, CrO₄^{2⊖}, BF₄^{⊖} and B(C₆H₅)₄^{⊖}.

In a further preferred embodiment of the process of the present invention, the substream of the solution of the stabilizer system is treated with oxygen prior to recirculation. The treatment with oxygen can be carried out at from 20 to 200° C., preferably from 50 to 170° C. and in particular from 100 to 150° C. The treatment with oxygen can advantageously be carried out using an oxygen-containing gas mixture, in particular a gas mixture consisting essentially of oxygen and nitrogen and having an oxygen content of from 3 to 10% by volume. A suitable oxygen-containing gas mixture is, for example, oxygen-depleted air. The treatment can be carried out at atmospheric pressure or superatmospheric pressure. The treatment with oxygen leads to effective regeneration of free N-oxyl radicals.

Apparatuses suitable for carrying out the oxygen treatment are all apparatuses which allow a liquid, in particular a viscous liquid, to be brought into contact with a gas, e.g. apparatuses for bubbling gas through a liquid, for injecting a gas stream into a liquid stream, etc. It is also possible to provide suitable mixing vessels, e.g. stirred mixing vessels. A plant suitable for carrying out the process of the present invention with oxygen treatment is shown in FIG. 2.

The invention will now be illustrated by the following examples.

### EXAMPLE 1 AND COMPARATIVE EXAMPLE 1

Crude styrene having the following composition was distilled at a rate of 100 kg/h in a distillation facility as shown in FIG. 1. A thin film evaporator was used as concentrator 4. A solution of the N-oxyl radical below in crude styrene was metered into the feed stream to the 1st column so that the concentration of N-oxyl radicals in the bottom of the 1st column was always in the range from 5 to 100 mg/kg.

Crude styrene composition:

1% by weight of benzene

2% by weight of toluene

40% by weight of ethylbenzene

56% by weight of styrene

1% by weight of high boilers.

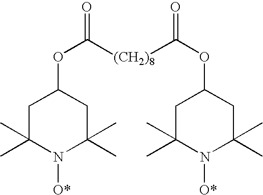

The high boiler fraction from the thin film evaporator was either discarded (Comparative Example 1) or a substream of 65% of the high boiler fraction was recirculated to the ethylbenzene column 2 (Example 1). The table below shows the viscosities and the styrene and polymer contents of the high boiler fraction from the thin film evaporator. The viscosity was measured using the Viscotester VT 02 from Haake Meβtechnik, Karlsruhe, Germany (nominal rotation speed 62.5 min⁻¹; rotating body 3). The polymer content was determined in accordance with ASTM D 2121-95.

It can be seen that in the case of recirculation of the high boiler fraction according to the present invention, the viscosities and polymer contents can be maintained at a stable, desirably low level. In Example 1 with recirculation of the high boiler fraction in accordance with the present invention, about 15% of N-oxyl stabilizer are saved compared to the comparative example without recirculation.

TABLE 1

| | Example 1 | | | | | Comparative Example 1 | | | | |
|---|---|---|---|---|---|---|---|---|---|---|
| | mPa s | | | Styr. | Poly | mPa s | | | Styr. | Poly |
| Days | 60° C. | 80° C. | 100° C. | % | % | 60° C. | 80° C. | 100° C. | % | % |
| Commencement | 400 | 220 | 180 | 9.7 | 5 | 540 | 280 | 150 | 7.7 | — |
| of the | | | | | | | | | | |
| observation | | | | | | | | | | |
| period | | | | | | | | | | |
| +36 | 800 | 380 | 210 | 3.3 | 5 | 625 | 310 | 175 | 4.7 | |
| +85 | 600 | 270 | 170 | 9.4 | 6 | 770 | 370 | 220 | 3.9 | |
| +106 | 630 | 280 | 140 | 8.7 | 4 | 800 | 410 | 250 | 2.6 | |
| +127 | 780 | 330 | 190 | 9.7 | 6 | 1300 | 800 | 475 | 2.7 | |
| +157 | 650 | 330 | 190 | 8.8 | 1250 | 700 | 450 | 2.9 | | |
| +197 | 380 | 190 | 120 | 9.4 | 600 | 340 | 200 | 2.8 | | |

### EXAMPLE 2 AND COMPARATIVE EXAMPLE 2

Example 1 was repeated, but the stabilizer used was an N-oxyl radical of the formula below in combination with an iron compound in the form of iron dibenzo[b,i]-1,4,8,11-tetraaza(14)annulene as activator. The N-oxyl radical and the iron compound were used in a weight ratio of 99.9:0.1.

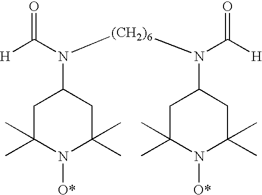

The viscosities and styrene and polymer contents of the high boiler fraction from the thin film evaporator are shown in Table 2 below.

TABLE 2

| | Example 2 | | | | | Comparative Example 2 | | | | |
|---|---|---|---|---|---|---|---|---|---|---|
| | mPa s | | | Styr. | Poly | mPa s | | | Styr. | Poly |
| Days | 60° C. | 80° C. | 100° C. | % | % | 60° C. | 80° C. | 100° C. | % | % |
| Commence- | 1050 | 650 | 450 | 6.7 | 6 | 900 | 480 | 300 | 4.1 | — |
| ment of the | | | | | | | | | | |
| oberservation | | | | | | | | | | |
| period | | | | | | | | | | |
| +7 | >1300 | 900 | 550 | 4.5 | 7 | 900 | 475 | 275 | 3.8 | — |
| +21 | 900 | 440 | 250 | 3.8 | 6 | | | | | — |
| +28 | 700 | 340 | 210 | 4.6 | 6 | 800 | 380 | 220 | 4.1 | — |
| +35 | 680 | 320 | 175 | 3.9 | 7 | 600 | 230 | 150 | 4.3 | — |
| +42 | 550 | 275 | 180 | 3.3 | — | 600 | 250 | 150 | 4.2 | — |
| +49 | 700 | 330 | 170 | 3.9 | — | 630 | 330 | 170 | 3.4 | — |
| +56 | 700 | 350 | 190 | 3.6 | — | 640 | 330 | 180 | 3.8 | — |
| +63 | 600 | 320 | 190 | 4.8 | — | 550 | 280 | 180 | — | — |
| +70 | 300 | 150 | 90 | 5.4 | — | 370 | 180 | 110 | 4.2 | — |
| +78 | 230 | 130 | 85 | 6.1 | — | 290 | 170 | 90 | 4 | — |
| +84 | 170 | 90 | 60 | 4.4 | — | 180 | 90 | 60 | 3.8 | — |

It can be seen that the viscosities and polymer contents were able to be lowered further compared to Example 1.

### EXAMPLE 3

Comparative Example 1 was repeated, but the stabilizer used was an N-oxyl of the following formula.

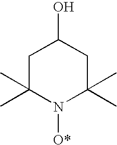

A sample of the high boiler fraction obtained in the thin film evaporator was taken and examined by ESR spectroscopy. The measurements were carried out using the laboratory version of the Miniscope MS 100 ESR spectrometer from Magnettech GmbH, Berlin, Germany. The instrument was calibrated beforehand using calibration solutions having known concentrations of N-oxyl radicals.

The sample was heated at 140° C. for 1 hour and the ESR measurement was repeated. The calculated contents of active N-oxyl radicals are shown in Table 3 below.

TABLE 3

| Sample | before heating | after heating |
|---|---|---|
| mg of N-oxyl radicals | 436 | 660 |

The results in Table 3 clearly show that the N-Oxyl radicals present in the high boiler fraction can be activated by heating to above 130° C.

We claim:

1. A process for the continuous recovery of styrene from a styrene-containing mixture by distillation of the mixture in a cascade of n distillation columns, where
(i) a feed stream comprising a stabilizer system is fed into the first column and/or a stabilizer system is introduced into at least one distillation column upstream of the nth distillation column, where the stabilizer system comprises N-oxyl radicals;
(ii) a high boiler fraction having a boiling point higher than that of styrene and comprising the stabilizer system accumulates in the bottom of the nth distillation column;
(iii) a substream of the high boiler fraction is recirculated and introduced into at least one distillation column upstream of the nth distillation column;
(iv) the remainder of the high boiler fraction is discharged from the process.

2. A process as claimed in claim 1, wherein the high boiler fraction comprises the relatively high-boiling components of the styrene-containing mixture and/or styrene oligomers.

3. A process as claimed in either of the preceding claims, wherein the high boiler fraction is concentrated prior to recirculation.

4. A process as claimed in any of the preceding claims, wherein the N-oxyl radicals pass through the (n−1)th distillation column an average of at least 1.4 times.

5. A process as claimed in any of the preceding claims, wherein the substream is heated to above 130° C. prior to recirculation.

6. A process as claimed in any of the preceding claims, wherein the stabilizer system further comprises a polymerization retarder.

7. A process as claimed in claim 6, wherein the polymerization retarder is an aromatic nitro compound.

8. A process as claimed in any of the preceding claims, wherein the stabilizer system further comprises an activator.

9. A process as claimed in claim 8, wherein the activator is an iron compound.

10. A process as claimed in any of the preceding claims, wherein the substream is treated with oxygen prior to recirculation.
